(19) United States
(12) Patent Application Publication
(10) Pub. No.: US 2024/0216575 A1
(43) Pub. Date: Jul. 4, 2024

(54) BIOPOLYMER SCAFFOLDS AND METHODS FOR PRODUCTION OR USE THEREOF

(71) Applicant: Bionova Medical, Inc., Germantown, TN (US)

(72) Inventor: Scott P. NOEL, Germantown, TN (US)

(73) Assignee: Bionova Medical, Inc., Germantown, TN (US)

(21) Appl. No.: 18/391,074

(22) Filed: Dec. 20, 2023

Related U.S. Application Data
(60) Provisional application No. 63/476,556, filed on Dec. 21, 2022.

(51) Int. Cl. A61L 27/20; A61L 27/24; A61L 27/54; C08L 5/04; C08L 5/08; C08L 89/06

(52) U.S. Cl. CPC A61L 27/20; A61L 27/24; A61L 27/54; C08L 5/04; C08L 5/08; C08L 89/06; A61L 2300/406; A61L 2430/34

(57) ABSTRACT

Methods for the manufacture of porous biopolymer scaffolds containing a first biopolymer in combination with chondroitin sulfate and/or hyaluronic acid, compositions made by such methods, and use of the biopolymer scaffolds in dermal regeneration. Non-limiting examples of first biopolymers include alginate, chitosan, collagen, cellulose, gelatin, starch, and various combinations thereof.

### CROSS-REFERENCE TO RELATED APPLICATIONS

This application claims priority to and the benefit of U.S. Provisional Application No. 63/476,556, filed Dec. 21, 2022, the entire contents of which is incorporated by reference herein.

### BACKGROUND

A subject having suffered skin loss or injury is threatened by infection and/or by loss of fluids. One method for addressing these threats and facilitating wound healing is through transplanting a partial-thickness section of skin to the wound. The transplanted section of skin may be obtained from an animal of another species or from a cadaver. However, such transplants are often rejected and can only serve to cover a wound for a short period of time. The transplanted section of skin can also be obtained from the subject. However, the process for obtaining the transplant is painful, invasive, can cause scarring, and, in some cases, the subject may not have sufficient skin available for transplant.

Given the limitations of skin transplantation, porous biopolymer scaffolds have been developed to facilitate wound healing as an alternative or precursor to skin transplantation. Such scaffolds allow for skin cell infiltration and creation of a new dermis to replace the membrane as it biodegrades. However, many porous biopolymer scaffolds available in the art suffer limitations such as, to name a few, potential for bleeding, infection, poor healing, poor structural integrity, or scarring.

Accordingly, there is a need for improved porous biopolymer scaffolds useful in facilitating wound healing.

### SUMMARY

The disclosure features methods for the manufacture of porous biopolymer scaffolds containing a first biopolymer in combination with chondroitin sulfate and/or hyaluronic acid, compositions made by such methods, and use of the biopolymer scaffolds in dermal regeneration. Non-limiting examples of first biopolymers include alginate, chitosan, collagen, cellulose, gelatin, starch, and various combinations thereof.

In one aspect, the disclosure features a biopolymer scaffold containing chondroitin sulfate and/or hyaluronic acid and chitosan, collagen, and/or alginate. If the biopolymer scaffold contains chondroitin sulfate and not hyaluronic acid, then the biopolymer scaffold does not contain collagen.

In another aspect, the disclosure features a multilayered medical device containing the biopolymer scaffold of any aspect provided herein, or embodiments thereof, and a polymer film.

In another aspect, the disclosure features a method for producing a biopolymer scaffold. The method involves (a) dissolving chondroitin sulfate and/or hyaluronic acid in an aqueous solution at about neutral pH. The method also involves (b) adding to the solution chitosan, collagen, and/or alginate and a combined volumetric percent of about 0.25% to about 2.0% of lactate and acetate. The method involves (c) lyophilizing the aqueous solution of (b) to produce a biopolymer scaffold. The method also involves (d) adjusting the pH of the biopolymer scaffold to a pH between about 6.0 to about 8.0. The method further involves (e) lyophilizing the biopolymer scaffold of (d) to produce a biopolymer scaffold.

In another aspect, the disclosure features a biopolymer scaffold produced by the method of any aspect provided herein, or embodiments thereof.

In another aspect, the disclosure features a method for treating a site of trauma in a subject. The method involves contacting the site with the biopolymer scaffold or multilayered device of any aspect provided herein, or embodiments thereof.

In another aspect, the disclosure features a kit for use in treatment of a wound containing the biopolymer scaffold or multilayered device of any aspect provided herein, or embodiments thereof.

In any of the aspects provided herein, or embodiments thereof, the biopolymer scaffold contains hyaluronic acid and chitosan. In any of the aspects provided herein, or embodiments thereof, the biopolymer scaffold contains chondroitin sulfate, and chitosan. In any of the aspects provided herein, or embodiments thereof, the biopolymer scaffold contains chitosan, hyaluronic acid, and chondroitin sulfate.

In any of the aspects provided herein, or embodiments thereof, the scaffold contains at least about 50% or more by weight chitosan, collagen, and/or alginate. In any of the aspects provided herein, or embodiments thereof, the scaffold contains at least about 75% or more by weight chitosan, collagen, and/or alginate. In any of the aspects provided herein, or embodiments thereof, the scaffold contains about 5-30% by weight chondroitin sulfate and/or about 1-10% hyaluronic acid. In any of the aspects provided herein, or embodiments thereof, the scaffold contains from about 10-25% chondroitin sulfate and/or about 3-8% hyaluronic acid.

In any of the aspects provided herein, or embodiments thereof, step (b) involves adding approximately equal volumetric percentages lactic acid and/or acetic acid.

In any of the aspects provided herein, or embodiments thereof, the aqueous solution of (a) contains 0.01-5% weight/weight (w/w) chondroitin sulfate and/or hyaluronic acid. In any of the aspects provided herein, or embodiments thereof, the aqueous solution of (a) contains 0.2% to 1% w/w chondroitin sulfate and/or hyaluronic acid. In any of the aspects provided herein, or embodiments thereof, the aqueous solution of (a) contains 0.1-5.0% chitosan, collagen, and/or alginate. In any of the aspects provided herein, or embodiments thereof, the aqueous solution of (a) contains about 0.5-1% chitosan, collagen, and/or alginate.

In any of the aspects provided herein, or embodiments thereof, the method further involves (f) contacting the lyophilized biopolymer scaffold with an aqueous solution containing a therapeutic agent followed by (g) lyophilizing the scaffold again.

In any of the aspects provided herein, or embodiments thereof, the therapeutic agent is selected from one or more of an anti-microbial agent, a growth factor, a hemostatic agent, an antithrombotic agent, cells, and an analgesic agent.

In any of the aspects provided herein, or embodiments thereof, the lyophilization step of (c) or (e) reduces the amount of water within the scaffold to less than about 5-10% of the weight of the scaffold. In any of the aspects provided herein, or embodiments thereof, the lyophilization step of (c) or (e) reduces the amount of water within the scaffold to less than about 1% of the weight of the scaffold.

In any of the aspects provided herein, or embodiments thereof, the site of trauma is a burn or wound. In any of the aspects provided herein, or embodiments thereof, the biopolymer scaffold is applied topically to the site of trauma.

In any of the aspects provided herein, or embodiments thereof, the biopolymer scaffold increases cellular infiltration of the site of trauma relative to a corresponding control subject that received conventional therapy.

In any of the aspects provided herein, or embodiments thereof, the biopolymer scaffold is infiltrated by cells at the site of trauma regrowth of tissue and neovascularization.

In any of the aspects provided herein, or embodiments thereof, the biopolymer scaffold further contains a therapeutic agent. In any of the aspects provided herein, or embodiments thereof, the therapeutic agent is selected from one or more of an anti-microbial, a growth factor, a hemostatic agent, an antithrombotic agent, cells, and an analgesic agent. In any of the aspects provided herein, or embodiments thereof, the anti-microbial is an iodine solution.

In any of the aspects provided herein, or embodiments thereof, the biopolymer scaffold and the anti-microbial are formulated separately and combined at a point of treatment.

The disclosure provides methods for the manufacture of porous biopolymer scaffolds containing a first biopolymer in combination with chondroitin sulfate and/or hyaluronic acid, compositions made by such methods, and use of the biopolymer scaffolds in dermal regeneration. Compositions and articles defined by the disclosure were isolated or otherwise manufactured in connection with the examples provided below. Other features and advantages of the invention will be apparent from the detailed description, and from the claims.

### Definitions

By “agent” is meant a polypeptide or polynucleotide, or fragments thereof, or a small molecule chemical compound.

By “alginate” or “alginic acid” is meant a compound containing homopolymeric blocks each having the structure

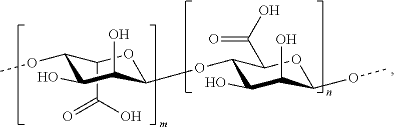

where “m” is any positive integer and “n” is any positive integer, where the blocks are covalently linked together in sequence, and pharmaceutically acceptable salts thereof.

By “biopolymer” is meant a polymer produced by or derived from an organism, a recombinant process, and/or a biosynthetic process. In an embodiment a biopolymer comprises chitosan, alginate, hyaluronic acid, and/or condroitin sulfate.

By “chitosan” is meant biopolymer comprising at least partially de-acetylated chitin. Preferably, chitosan is at least about 50% deacetylated. Chitin is a linear polysaccharide comprising (1-4)-linked 2-acetamido-2-deoxy-b-D-glucopyranose. Chitosan is a linear polysaccharide consisting of (1-4)-linked 2-amino-2-deoxy-b-D-glucopyranose.

By “chondroitin sulfate” or “sulfated glycosaminoglycan (GAG)” is meant a compound having the structure

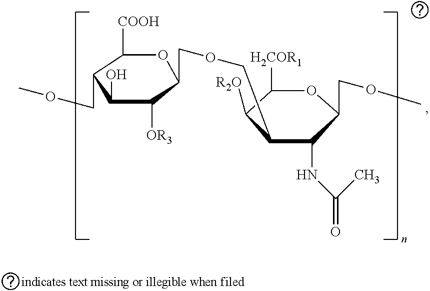

where “n” is any positive integer, and either R₁ is H, R₂ is SO₃H and R₃ is H or R₁ is SO₃H, R₂ is H and R₃ is H, and pharmaceutically acceptable salts thereof. In embodiments, n is about or at least about 10, 20, 30, 40, 50, 75, 100, 200, 300, 400, 500, 600, 700, 800, 900, 1000, 5000, 10000, or greater. In embodiments, n is less than about 10, 20, 30, 40, 50, 75, 100, 200, 300, 400, 500, 600, 700, 800, 900, 1000, 5000, 10000.

By “hyaluronic acid” or “Poly {[(2S,3R,4R,5S,6R)-3-acetamido-5-hydroxy-6-(hydroxymethyl)oxane-2,4-diyl]oxy[(2R,3R,4R,5S,6S)-6-carboxy-3,4-dihydroxyoxane-2,5-diyl]oxy}” is meant a compound having the structure

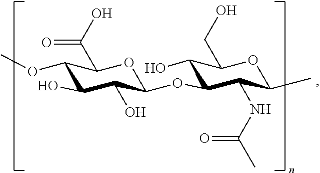

where “n” is any positive integer, and corresponding to CAS No. 9004-61-9, and pharmaceutically acceptable salts thereof. In embodiments, n is about or at least about 10, 20, 30, 40, 50, 75, 100, 200, 300, 400, 500, 600, 700, 800, 900, 1000, 5000, 10000, or greater. In embodiments, n is less than about 10, 20, 30, 40, 50, 75, 100, 200, 300, 400, 500, 600, 700, 800, 900, 1000, 5000, 10000.

By “agent” is meant any small compound, molecule, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) as detected by standard art known methods such as those described herein.

By “analog” is meant a molecule that is not identical but has analogous functional or structural features. For example, a chitosan analog retains the biological activity of a corresponding reference chitosan polymer (e.g., manufactured chitosan), while having certain biochemical modifications that enhance the analog's function relative to a reference chitosan polymer. Such biochemical modifications may increase the analog's ability to be degraded, to uptake or elute a therapeutic agent, or to increase or decrease mechanical strength.

By “antimicrobial” is meant an agent that inhibits or stabilizes the proliferation or survival of a microbe. In one embodiment, a bacteriostatic agent is an antimicrobial. In other embodiments, any agent that kills a microbe (e.g., bacterium, fungus, virus) is an antimicrobial. A non-limiting example of an antimicrobial is an antibiotic (e.g., vancomycin) or iodine. In some embodiments, the iodine is molecular iodine (i.e., I₂) or an iodine ion.

By “anti-inflammatory” is meant an agent that reduces the severity or symptoms of an inflammatory reaction in a tissue. An inflammatory reaction within tissue is generally characterized by leukocyte infiltration, edema, redness, pain, and/or neovascularization. Inflammation can also be measured by analyzing levels of cytokines or any other inflammatory marker.

By “biodegradable” is meant susceptible to breakdown by biological activity. For example, biodegradable scaffolds are susceptible to breakdown by enzymes present in vivo (e.g., lysozyme, N-acetyl-o-glucosaminidase and lipases). Degradation of a scaffold of the disclosure need not be complete. A scaffold of the disclosure may be degraded, for example, by the cleavage of one or more chemical bonds (e.g., glycosidic bonds). Advantageously, degradation is by at least about 20, 30, 40, 50, 60, 70, 80, 90, 95% or more over 3 to 5, 5 to 7, 7 to 9, 10 to 15, or 15 to 30 days.

In this disclosure, “comprises,” “comprising.” “containing” and “having” and the like can have the meaning ascribed to them in U.S. Patent law and can mean “includes,” “including,” and the like: “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “degrades” is meant physically or chemically breaks down in whole or in part. Preferably, the degradation represents a physical reduction in the mass by at least about 10%, 25%, 50%, 75%, 80%, 85%, 90%, 95% or 100%.

By “degradation rate” is meant the time required to substantially degrade the composition. A composition is substantially degraded where at least about 20%, 30%, 40%, 50%, 60%, 75%, 85%, 90%, 95% or more has been degraded. Methods for measuring degradation of chitosan are known in the art and include measuring the amount of a composition (e.g., a scaffold) of the disclosure that remains following implantation in a subject or following in vitro exposure to an enzyme having chitosan-degrading activity.

“Detect” refers to identifying the presence, absence or amount of the analyte to be detected.

By “disease” is meant any condition or disorder that damages or interferes with the normal function of a cell, tissue, or organ. In one example, a disease is a bacterial or other infection present in a wound site. In another embodiment, a disease is sepsis.

By “effective amount” is meant the amount of an agent required to ameliorate the symptoms of a disease relative to an untreated patient. The effective amount of active agent(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “elution rate” is meant the time required for an agent to be substantially released from a composition. Elution can be measured by determining how much of an agent remains within the composition or by measuring how much of an agent has been released into the composition's surroundings. Elution may be partial (e.g., 10%, 25%, 50%, 75%, 80%, 85%, 90%, 95% or more) or complete. In one embodiment, the agent continues to be released at an effective level for at least about 3 days, 4 days, 5 days, 6 days, 7 days, 8 days, 9 days, 10 days, 3 weeks, 4 weeks, 5 weeks, 6 weeks, 7 weeks, 8 weeks, 9 weeks, 10 weeks, or longer.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “infection” is meant the presence of one or more pathogens in a tissue or organ of a host. An infection includes the proliferation of a microbe (e.g., bacteria, viruses, fungi) within a tissue of a subject at a site of trauma. By “modulate” is meant alter. In embodiments, an alteration is an increase or decrease. Such alterations are detected by standard art known methods such as those described herein.

As used herein, “obtaining” as in “obtaining an agent” includes synthesizing, purchasing, or otherwise acquiring the agent.

By “physical interaction” is meant an association that does not require covalent bonding. In one embodiment, a physical interaction includes incorporation into a scaffold of the disclosure.

By “point of treatment” is meant the site where healthcare is delivered. A “point of treatment” includes, but is not limited to, a surgical suite, physician's office, clinic, or hospital.

By “polymer” is meant a natural or synthetic organic molecule formed by combining smaller molecules. In embodiments, the polymer comprises 2, 3, 4, 5, 6, 7, 8, 9, 10 or more of the smaller molecules joined to one another by covalent bonds.

By “profile” is meant a set of characteristics that define a composition or process. For example, a “biodegradation profile” refers to the biodegradation characteristics of a composition. In another example, an “elution profile” refers to elution characteristics of a composition. By “small molecule” is meant any chemical compound.

By “scaffold” is meant a three-dimensional porous matrix. In some embodiments, the scaffold serves as a substrate for cell growth or vascular infiltration.

By “trauma” is meant any injury that damages a tissue or organ of a subject. The injury need not be severe. Therefore, a trauma includes any injury that breaks the skin.

By “modulation” is meant any alteration (e.g., increase or decrease) in a biological function or activity.

By “subject” is meant a mammal. Non-limiting examples of mammals include a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition. In one embodiment, the reference is a site of trauma in a control subject that is not treated or that is treated with conventional therapy (i.e., the wound is covered with a gauze pad). In an embodiment, the reference is a scaffold not manufactured according to the methods provided herein or lacking a component (e.g., chondroitin sulfate and/or hyaluronic acid) of a scaffold provided herein.

By “wound management device” or “wound healing device” is meant any material used to protect or promote healing at a site of trauma

### DETAILED DESCRIPTION

As described below, the present disclosure features, among other things, methods for the manufacture of biopolymer scaffolds containing a first biopolymer in combination with chondroitin sulfate and/or hyaluronic acid, compositions made by such methods, and use of the biopolymer scaffolds in dermal regeneration. Non-limiting examples of biopolymers include alginate, chitosan, collagen, cellulose, gelatin, starch, and various combinations thereof.

The invention is based, at least in part, on the discovery that a first biopolymer (e.g., chitosan) can be dissolved in an approximately pH neutral solution containing dissolved chondroitin sulfate and/or hyaluronic acid by adding the pH neutral solution one or more acids (e.g., lactic acid and acetic acid). Accordingly, the present disclosure provides, among other things, methods for preparing biopolymer compositions (e.g., wound management device) containing a first biopolymer (e.g., chitosan) and chondroitin sulfate and/or hyaluronic acid.

In embodiments, the scaffolds are biodegradable compositions containing a biopolymer (e.g., chitosan) and chondroitin sulfate and/or hyaluronic acid. In some embodiments, the biopolymer scaffolds of the disclosure contain and/or provide for the local delivery of a therapeutic agent (e.g., antimicrobial, cells, analgesics, and/or any agent listed herein or combinations thereof). In embodiments, the disclosure features methods of using a scaffold provided herein to treat or prevent a site of trauma. In embodiments, the biopolymer scaffold protects from or treats an infection and/or promotes healing at the site of trauma.

### Chitosan

Chitosan is a linear polysaccharide composed of randomly distributed ß-(1-4)-2-amino-2-D-glucosamine (deacetylated) and ß-(1-4)-2-acetamido-2-D-glucoseamine (acetylated) units. Chitosan is derived from chitin, a naturally occurring polymer. Chitin is a white, hard, inelastic, nitrogenous polysaccharide isolated from fungi, mollusks, or from the exoskeletons of arthropods (e.g., crustaceans, insects). The major procedure for obtaining chitosan is the alkaline deacetylation of chitin with strong alkaline solution. Generally, the raw material is crushed, washed with water or detergent, and ground into small pieces. After grinding, the raw material is treated with alkali and acid to isolate the polymer from the raw crushed material. The polymer is then deacetylated by treatment with alkali. Chitin and chitosan differ in their degrees of deacetylation (DDA). Chitin has a degree of deacetylation of 0% while pure chitosan has a degree of deacetylation of 100%. Typically, when the degree of deacetylation is greater than about 50% the polymer is referred to as chitosan.

Chitosan is a cationic weak base that is substantially insoluble in water and organic solvents. Typically, chitosan is fairly soluble in dilute acid solutions, such as acetic, citric, oxalic, proprionic, ascorbic, hydrochloric, formic, and lactic acids, as well as other organic and inorganic acids. Chitosan's charge gives it bioadhesive properties that allow it to bind to negatively charged surfaces, such as biological tissues present at a site of trauma, negatively charged implanted devices, or to charged or polar compounds (e.g., molecular iodine (12) or an iodine ion, or a polymer, such as, glycosaminoglycan). Chitosan's degree of deacetylation affects it resorption. Chitosan compositions having a 50% degree of deacetylation are highly degradable in vivo. As the degree of deacetylation increases, chitosan becomes increasingly resistant to degradation. Chitosan compositions having a degree of deacetylation that is higher than 95% degrade slowly over weeks or months. In the body chitosan is degraded by lysozyme, N-acetyl-o-glucosaminidase and lipases. Lysozyme degrades chitosan by cleaving the glycosidic bonds between the repeating chitosan units. The byproducts of chitosan degradation are saccharides and glucosamines that are gradually absorbed by the human body. Therefore, when chitosan is used for the local delivery of therapeutic or prophylactic agents, no secondary removal operation is required.

Crystallinity indicates the degree of structural order in a compound. Polymers such as chitosan are either amorphous or semicrystalline. Chitosan's crystallinity, like other polymers, depends on its type, number, and regularity of polymer-chain, side group chemistry, the degree of matrix packing or density, and crosslinking. The crystallinity of chitosan or its products can be controlled or altered during manufacture through its molecular weight, degree of deacetylation, and crosslinking to affect thermal properties, such as melting point, and physical-mechanical properties, such as tensile strength, Young's modulus, swelling and degradation.

Crosslinking is the process which links polymer chains together. In chitosan, crosslinking induces a three-dimensional matrix of interconnected, linear, polymeric chains. The degree or extent of crosslinking depends on the crosslinking agent. Exemplary crosslinking agents include sodium tripolyphosphate, ethylene glycol diglycidyl ether, ethylene oxide, glutaraldehyde, epichlorohydrin, diisocyanate, and genipin. Crosslinking can also be accomplished using microwave or ultraviolet exposure.

Chitosan's properties can also be altered by modulating the degree of deacetylation. In one embodiment, the degree of deacetylation is adjusted between about 50-100%, wherein the bottom of the range is any integer between 50 and 99, and the top of the range is any integer between 51% and 100%. In particular embodiments, the degree of deacetylation is 51%, 55%, 60%, 61%, 65%, 70%, 71%, 73%, 74%, 75%, 76%, 77%, 78%, 79%, 80%, 85%, 90%, and 95%. In general, the higher the degree of deacetylation, the slower the degradation of the chitosan composition. In embodiments, the degree of deacetylation of the chitosan is between about 70% and about 80%.

### Methods for Preparation

In various aspects, the present disclosure provides methods for producing a biopolymer scaffold. The biopolymer scaffold comprises a first biopolymer and one or both of chondroitin sulfate and hyaluronic acid. Non-limiting examples of first biopolymers include alginate, chitosan, collagen, cellulose, gelatin, starch, and various combinations thereof. In some embodiments, the base polymer is chitosan. In some instances, the base polymer is collagen.

In embodiments, the methods of the disclosure involve dissolving chondroitin sulfate and/or hyaluronic acid in a solution. Typically, the solution has a near-neutral pH (e.g., between 6 and 8 (e.g., 6, 6.5, 7, 7.5, 8), between 6.5 and 7.5 (e.g., 6.5, 6.6, 6.7, 6.8, 6.9, 7.0, 7.1, 7.2, 7.3, 7.4, 7.5), or about 7) at ambient temperature (e.g., 25° C.). The methods further involve adding a first biopolymer, such as chitosan, to the solution containing the dissolved chondroitin sulfate and/or hyaluronic acid. The order in which chondroitin sulfate, hyaluronic acid, and base polymer are added to the solution is not limiting. However, in embodiments, chondroitin sulfate and/or hyaluronic acid are dissolved in the near-neutral pH solution prior to dissolution of the first biopolymer. In embodiments, the first biopolymer is added to a solution after hyaluronic acid and/or chondroitin sulfate have been dissolved in the solution. In embodiments, the solution is an aqueous solution containing deionized, distilled, or distilled-deionized water.

In embodiments, a solution of the disclosure contains about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, or 10.0 g/L of hyaluronic acid. In embodiments, the solution no more than about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, or 10.0 g/L of hyaluronic acid.

In embodiments, a solution of the disclosure contains about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, or 10.0 g/L of chondroitin. In embodiments, the solution no more than about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, or 10.0 g/L of chondroitin.

In embodiments, a solution of the disclosure contains about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, 6.0 g/L, 7.0 g/L, 8.0 g/L, 9.0 g/L, 10.0 g/L, 15.0 g/L, 20.0 g/L, or 50.0 g/L of the first biopolymer (e.g., chitosan). In embodiments, the solution no more than about, or at least about 0.1 g/L, 0.2 g/L, 0.3 g/L, 0.4 g/L, 0.5 g/L, 0.6 g/L, 0.7 g/L, 0.8 g/L, 0.9 g/L, 1.0 g/L, 2.0 g/L, 3.0 g/L, 4.0 g/L, 5.0 g/L, 6.0 g/L, 7.0 g/L, 8.0 g/L, 9.0 g/L, 10.0 g/L, 15.0 g/L, 20.0 g/L, or 50.0 g/L of the first biopolymer (e.g., chitosan).

In one embodiment, the mass ratio of the first biopolymer to hyaluronic acid in the solution is about, or at least about 2:1, 3:1, 4:1, 5:1, or 6:1. In one embodiment, the mass ratio of the first biopolymer to hyaluronic acid in the solution is less than about 2:1, 3:1, 4:1, 5:1, or 6:1. In some cases, the mass ratio of the first biopolymer to chondroitin sulfate in the solution is about, or at least about 3:1, 4:1, 5:1, 6:1, 7:1, 8:1, 9:1, 10:1, 11:1, 12:1, 13:1, or 14:1. In some cases, the mass ratio of the first biopolymer to chondroitin sulfate in the solution is less than about 3:1, 4:1, 5:1, 6:1, 7:1, 8:1, 9:1, 10:1, 11:1, 12:1, 13:1, or 14:1. In some instances, the mass ratio of the first biopolymer to the combined mass of hyaluronic acid and chondroitin sulfate in the solution is about, or at least about 3:1, 4:1, 5:1, 6:1, 7:1, 8:1, 9:1, 10:1, 11:1, 12:1, 13:1, or 14:1. In some instances, the mass ratio of the first biopolymer to the combined mass of hyaluronic acid and chondroitin sulfate in the solution is less than about 3:1, 4:1, 5:1, 6:1, 7:1, 8:1, 9:1, 10:1, 11:1, 12:1, 13:1, or 14:1. In some cases, the mass ratio of chondroitin sulfate to hyaluronic acid in the solution is about, or at least about, 1:1, 2:1, 3:1, 4:1, or 5:1. In some cases, the mass ratio of chondroitin sulfate to hyaluronic acid in the solution is less than about, 1:1, 2:1, 3:1, 4:1, or 5:1.

Once the chondroitin sulfate and/or hyaluronic acid are dissolved in the solution and the first biopolymer has been added to the solution, the methods of the disclosure involve then dissolving the first biopolymer in the solution by adding one or more acidic solutions (e.g., aqueous solutions) each containing one or more acids (e.g., weak organic acids) to the solution until the first biopolymer (e.g., chitosan) dissolves in the solution. In embodiments, the acidic solutions each contain one or more of acetic acid, lactic acid, citric acid, propionic acid, hydrochloric acid, sulfuric acid, or any acid known in the art. In some cases, the acidic solutions contain a weak organic acid. In embodiments, acetic acid is used to produce a scaffold with increased strength and slower degradation as compared to a scaffold produced using an alternative acid and not acetate.

In various embodiments, combinations of acetic and lactic acids are used. In one approach, the ratio of lactic to acetic acid added to the solution is about 5:1, 4:1, 3:1, 2:1, 1:1, 1:2, 1:3, 1:4, to 1:5.

The acidic solutions may each individually contain a total concentration of about or at least about 0.1% v/v, 0.2% v/v, 0.3% v/v, 0.4% v/v, 0.5% v/v, 0.6% v/v, 0.8% v/v, 0.9% v/v, 1% v/v, 2% v/v, 3% v/v, 4% v/v, or 5% v/v acid. In some cases, the methods of the disclosure involve adding two or more acidic solutions to the solution containing the first biopolymer (e.g., one solution containing lactate and one solution containing acetate). If more than one acidic solution is added to the solution containing the first biopolymer, the methods of the disclosure may involve adding approximately equal total amounts (e.g., equal volumes or equal masses) of each acidic solution. The acidic solutions may each contain similar or equal concentrations of acid.

In embodiments, the final concentration of acid in the solution containing the first biopolymer is about or at least about 0.01 v/v, 0.1 v/v, 0.2 v/v, 0.3 v/v, 0.4 v/v, 0.5 v/v, 0.6 v/v, 0.7 v/v, 0.8 v/v, 0.9 v/v, 1.0 v/v, 1.25 v/v, 1.5 v/v, 1.75 v/v, 2.0 v/v, 3.0 v/v, 4.0 v/v, or 5.0 v/v. In embodiments, the final concentration of acid in the solution containing the first biopolymer is less than about 0.1 v/v, 0.2 v/v, 0.3 v/v, 0.4 v/v, 0.5 v/v, 0.6 v/v, 0.7 v/v, 0.8 v/v, 0.9 v/v, 1.0 v/v, 1.25 v/v, 1.5 v/v, 1.75 v/v, or 2.0 v/v.

In various instances, the solution containing dissolved chondroitin sulfate and/or hyaluronic acid is a gel. In some instances, the solution containing the dissolved first biopolymer is a gel.

In some cases, the solution containing chondroitin sulfate, hyaluronic acid, and/or the first biopolymer is heated to assist in dissolution of one or more of the compounds. In embodiments, the solution is heated to about, or at least about 10° C., 15° C., 20° C., 25° C., 26° C., 27° C., 28° C., 29° C., 30° C., 31° C., 32° C., 33° C., 34° C., 35° C., 36° C., 37° C., 38° C., 39° C., 40° C., 45° C., 50° C., 55° C., 60° C., 65° C., or 70° C. In embodiments, the solution is heated to no more than 10° C., 15° C., 20° C., 25° C., 26° C., 27° C., 28° C., 29° C., 30° C., 31° C., 32° C., 33° C., 34° C., 35° C., 36° C., 37° C., 38° C., 39° C., 40° C., 45° C., 50° C., 55° C., 60° C., 65° C., or 70° C. In some embodiments, the solution is kept at a temperature (e.g., about 40° C.) to keep the resulting solution workable with its high viscosity (e.g., allowing the solution to lie flat in a tray).

In embodiments, the methods involve mixing the solution containing the chondroitin sulfate, the hyaluronic acid, and/or the first biopolymer to facilitate dissolution of the compounds. Once the first biopolymer is dissolved in the solution with the chondroitin sulfate and/or hyaluronic acid, the resulting solution is mixed for about, or at least about 10 min, 20 min, 30 min, 1 hr, 2, hr, 3 hr, 4 hr, 5 hr, 6 hr, 7 hr, 8 hr, 9 hr, 10 hr, 11 hr, 12 hr, or 24 hr.

In embodiments, the methods of the disclosure involve casting into a mold or tray the solutions containing dissolved chitosan in combination with one or more of dissolved hyaluronic acid and chondroitin sulfate.

The methods provided herein further involve freezing the solutions containing dissolved chitosan and one or more of dissolved hyaluronic acid and chondroitin sulfate followed by lyophilization for a set number of hours to produce a dehydrated scaffold. In embodiments, the solutions are lyophilized for about or at least about 1 hr, 2 hr, 3 hr, 4 hr, 5 hr, 6 hr, 12 hr, 1 day, 2 days, 3 days, 4 days, 5 days, or 6 days. In embodiments, the solutions are lyophilized for no more than about 1 hr, 2 hr, 3 hr, 4 hr. 5 hr, 6 hr, 12 hr, 1 day, 2 days, 3 days, 4 days, 5 days, or 6 days. In embodiments, the methods involve lyophilizing the scaffold an additional 1, 2, 3, 4, 5, or more times. Lyophilization is conducted to reduce the liquid (e.g., water) content of the matrix to less than about 5%, 10%, 20%, 25%, 30%, 40%, 50%, 75%, 80%, 90%, 95%, or 100% by weight. Typically, lyophilization removes at least about 70%, 75%, 80%, 90%, 95, or 100% or the original water content of the scaffold. Scaffolds that retain some moisture may be packaged in sterile foil to maintain such moisture. In various embodiments, the total times a scaffold is lyophilized according to the methods of the disclosure does not exceed 1, 2, 3, 4, 5, 6, 7, 8, 9, or 10.

In embodiments, the scaffold is neutralized following lyophilization. In various cases, neutralization involves hydrating the lyophilized scaffold in an excess concentration and volume of a basic solution (e.g., sodium hydroxide aqueous solution). The residual acidic and basic products are then washed away using water (e.g., ultrapure water) to bring the pH of the scaffold to approximately neutral (e.g., pH of between 6 and 7, or of about 7). Following neutralization, the scaffold may be lyophilized one or more times again, as described above. Any base known to the skilled practitioner (e.g., NaOH, KOH, NH₄OH, Ca(OH)₂, Mg(OH)₂, or combinations thereof) may be used to neutralize the scaffold. In some cases, a neutralization solution has a pH greater than 7.4 (e.g., 7.8, 8.0, 8.5, 9.0, 10, 11, and 12, 13, 14, 15, 16). The neutralization step may be omitted. If desired, the scaffold is treated with water, PBS, or sterile saline. The basic solution may comprise from about 0.01 to about 10.0M of a base (e.g., 0.01, 0.025, 0.5, 0.75, 0.1, 0.25, 0.5, 0.75, 1.0, 1.5, 2.0, 2.5, 3, 4, 5, 6, 7, 8, 9, 10 M) (e.g., NaOH). In some cases, scaffolds neutralized in bases having lower molarity degrade more quickly. Accordingly, a lower molarity base may be used to produce a scaffold that degrades more quickly. Scaffolds neutralized in bases of increased molarity degrade more slowly than those neutralized at lesser molarities. Accordingly, a higher molarity base may be used to produce a scaffold that degrades more slowly. Therefore, the degradation properties of chitosan may be modulated by altering the molarity of the neutralizing base. Neutralization may be followed by one or more lyophilization steps carried out as described above.

In various embodiments, preparation of a scaffold involves at least two lyophilization steps. In some instances, the first lyophilization is the lyophilization of the solution, as described above, to yield a scaffold, and the second lyophilization is the lyophilization following neutralization of the scaffold, as described above (2 Lyo method). In embodiments, preparation of a scaffold using this 2 Lyo method reduces degradation and/or improves stability of a scaffold relative to a scaffold prepared using only the first lyophilization (i.e., no neutralization: 1 Lyo method). Representative 2 Lyo methods include those described in, e.g., U.S. Patent Application Publication No. 2017/0258967, the disclosure of which is incorporated herein by reference in its entirety for all purposes. In some cases, the 2 Lyo method improves elution of an agent from the scaffold relative to a scaffold prepared using an alternative method (e.g., the 1 Lyo method). In some instances, scaffolds prepared using the 2 Lyo method exhibit increased crystallinity relative to scaffolds prepared using the 1 Lyo method or alternative methods not involving any neutralization step. In some cases, a scaffold prepared by the 2 Lyo method has increased porosity relative to a scaffold prepared using the 1 Lyo method or an alternative method not including any neutralization step.

In some cases, the methods of the disclosure include incubating the scaffold in a buffer, where the buffer has a pH between 2.5 and 6.5 (e.g., a pH of about 2.5, 3.0, 4.0, 5.0, 5.1, 5.2, 5.3, 5.4, 5.5, 5.6, 5.7, 5.8, 5.9, and 6.0). In certain embodiments, the buffer is sodium or potassium acetate, bicarbonate, carbonate, citrate, formate, glycine, malate, maleate, 2-(N-morpholino)ethanesulfonate, phosphate, proprionate, or succinate buffer. The buffering treatment is optimized to optimize the degradation profile of the scaffold. Time of buffering treatment may be from as little as 30 seconds to as long as 12 hours (e.g., 1, 3, 5, 10, 15, 30, 45, 60 minutes, 1, 3, 5, 10, 12 hours). The concentration of the buffer may be varied between about 0.05 and 2.0 molar. Preferably, such variables are adjusted such that the scaffold degrades by at least about 20%, 30%, 40%, 50%, 60% or more in 3, 5, 7 or 10-days. Buffering may be followed by one or more lyophilization steps carried out as described above.

Or the scaffolds can have one or more additional components added to them (e.g., iodine). The one or more components may be added at any point during the preparation of the scaffolds. For example, a component may be added during the dissolution of chondroitin sulfate, hyaluronic acid, or the first biopolymer in the solution. In some embodiments, the components are added by contacting the scaffold with a solution (e.g., an aqueous solution) containing the components followed by one or more lyophilization steps, as described above. Non-limiting examples of components include antimicrobial agents (i.e., silver, chlorhexidine, iodine, etc.), antibiotics (i.e., vancomycin, tobramycin, gentamicin, meropenam, cefazolin, etc.), antifungal agents (i.e., amphotericin B, fluconazole, voriconazole, etc.), analgesics (i.e., lidocaine, bupivacaine, morphine, etc.), anti-inflammatoires (i.e., indomethacin, toradol, kenalog, etc.), cellular therapies/growth factors (i.e., amniotic tissue, amniotic liquid, platelet-rich plasma, IGF, VEGF, BMP, multipotent cells (e.g., bone marrow cells, stem cells), plasma, skin seeds, morselized skin fragments, morselized dermal tissue, etc.), cell conditioned media, collagen, omega 3 fatty acids, and various combinations thereof. In some embodiments, one or more lyophilization steps may be carried out several times as needed to get the appropriate quantity of additional components into the device. The total number of freeze-dry cycles may be, for example, 1, 2, 3, 4, 5, 6, 7, 8, 9, 10, or more. In embodiments, a scaffold prepared as described herein and containing the one or more additional components (e.g., iodine) is lyophilized a total of three times. In various cases, the one or more additional components are added after neutralization of the scaffold as described above.

A scaffold may be contacted with a therapeutic agent (e.g., iodine) such that the agent is incorporated into the chitosan. This contacting may be carried out before or during a procedure to treat a subject using methods described herein. In some embodiments, a scaffold prepared as described above is contacted with an iodine composition followed by a further lyophilization (e.g., a third lyophilization).

A third (or subsequent) lyophilization step may be advantageous in maintaining performance of embedded agents (3 Lyo method). For example, iodine can react with sodium hydroxide (a neutralizing agent), such as sodium hydroxide used in the neutralization described above, to remove bonds between chitosan and iodine and form sodium iodide and sodium iodate complexes. It can, therefore, be advantageous to prepare a scaffold by first lyophilizing a solution, as described above, to yield a scaffold, to then treat the scaffold to neutralize the scaffold as described above and then lyophilize the now-neutralized scaffold a second time prior to contacting the scaffold with an agent (e.g., molecular iodine, any agent described herein or combinations thereof) followed by a third lyophilization step. This three-lyophilization process (3 Lyo method) has the advantage of avoiding contacting iodine, or any alternative agent or combination thereof administered after the second lyophilization step, with sodium hydroxide or any other agent used during the neutralization step. Some agents (e.g., pharmaceutical agents) may undergo inactivation or denaturing if exposed to the base/acid neutralization process. Addition of an antimicrobial, antiseptic, anticoagulant, hemostatic agent, and/or antithrombotic agent(s) may be added after the second lyophilization step to avoid potential deactivation issues. Incorporation of agents into a scaffold achieved using this three-lyophilization method may reduce or eliminate the need to add them at a point of treatment.

In embodiments, the scaffold is stabilized structurally and remains in a dense and compacted state until contacted with a liquid susceptible to absorption by the scaffold, for example, body fluids. For medical use, the compacted or compressed scaffold is sterilized using any suitable means (e.g., radiation, ethylene oxide (e.g., ethylene oxide gas), vaporized hydrogen peroxide, and/or ethanol treatment). The scaffold may be packaged in sterile packaging for medical use. Scaffold elements or other devices of the disclosure may also contain one or more active therapeutic agents, such as those described herein. For example, they include agents that promote clotting (e.g., thrombin and/or fibrinogen). Alternatively, or in addition, a scaffold the disclosure includes antibiotics and/or growth factors that promote tissue growth and healing.

In various embodiments, a scaffold may be die-cut into individual units suitable for use in treating a wound. In other embodiments, the scaffolds may also be molded into smaller units without cutting (pre-fabricated molds of appropriate size). The scaffolds may be used as stand-alone products to help regenerate damaged or missing tissue(s) or they may be combined with any one or more of the additional components (e.g., aqueous components) provided herein.

In some embodiments, a scaffold may be ground into smaller particles and used alone as a powder to administered to a wound, or may be combined with 1, 2, 3, 4, or 5 similarly fabricated and ground scaffolds. This powder may be used without hydrating in an aqueous solution or may be combined with any one or more of the additional components (e.g., aqueous components) provided herein. The powder may contain only particles with a size of no larger than about 0.5 microns, 1 micron, 10 microns, 25 microns, 50 microns, 100 microns, 500 microns, 1000 microns, 15000 microns, or 2000 microns. The powder may contain particles with a size of only less than about 0.5 microns, 1 micron, 10 microns, 25 microns, 50 microns, 100 microns, 500 microns, 1000 microns, 15000 microns, or 2000 microns. Various methods are known in the art for preparing powders (e.g., mechanical crushing). Powders may be using mechanical forces such as crushing (e.g., pulverizing, rolling, and jawing), striking (e.g., with hammer or similar tools), or grinding (with ball and rod or a blender). Preparation of a powder can involve sifting of a sample to isolate particles falling within a particular size range (i.e., particles having a particular range of diameters). Fine powders may be produced using hammer mills, rod mills, normal ball mills, vibration ball mills, or stirring ball mills. In the ball milling process, the balls may be made of corundum, with great hardness and strength, and the ball milling can take place in air or in a liquid, such as water, alcohol, gasoline, or acetone.

In embodiments, scaffolds produced by the methods provided herein are reproducibly and uniformly degradable. In embodiments, the degradation process may be controlled by varying the process used to manufacture the scaffold.

A scaffold may be loaded with a biologically active agent at a site of care (e.g., in a surgical suite, clinic, or physician's office, trauma site, battlefield). This allows a clinician to tailor the antibiotics or other agents used to load the chitosan wound management device to suit the needs of a particular patient. In one embodiment, the degree of deacetylation of chitosan is adjusted to provide scaffolds containing the chitosan that degrade in as little as about twenty-four, thirty-six, forty-eight, or seventy two hours or that are maintained for a longer period of time (e.g., 4, 5, 6, 7, 8, 9, 10 days). In other embodiments, scaffolds of the disclosure are maintained in a subject for at least about two to six weeks or more (e.g., 2, 3, 4, 5, 6 weeks, two, three or four months). In still other embodiments, scaffolds of the disclosure enhance blood clotting and/or vascularization in a wound or other site of trauma. In embodiments, the scaffolds are loaded with therapeutic or prophylactic agents that are clinician selected and that are delivered over at least about 1, 2, 3, 4, 5, 6, 7, 8, 9, or 10 days or for longer periods.

### Biopolymer Scaffolds

In various aspects, the disclosure provides biopolymer scaffolds produced according to the methods provided herein. In an embodiment the scaffold contains a first biopolymer and one or more of hyaluronic acid and chondroitin sulfate. Non-limiting examples of the first biopolymer include alginate, chitosan, collagen, cellulose, gelatin, starch, and various combinations thereof.

Scaffolds of the disclosure may advantageously be expandable when wetted. In some embodiments, the scaffold has the capacity to expand at least about 10%, 20%, 30%, 40%, 50%, 50%, 60%, 70%, 80%, 90%, 100%, or more when wetted. In other embodiments, a scaffold expands by about 200% by volume when wetted to saturation with deionized water, buffer, or an agent of the disclosure. Scaffolds may achieve rapid volume expansions (e.g., when immersed in aqueous solution).

Scaffolds may be produced in any size required for application to a wound. In one embodiment, an expanded scaffold exerts compression on surrounding tissues when implanted or delivers an active agent to the implantation site and surrounding tissue. Advantageously, scaffold compositions generated with a buffering step described herein provide for the controlled degradation of the scaffolds, and/or provide for the controlled release one or more agents contained in the scaffold.

In some instances, a scaffold of the disclosure contains about, or at least about 50 wt %, 55 wt %, 60 wt %, 65 wt %, 70 wt %, 75 wt %, 80 wt %, 85 wt %, 90 wt %, or 95 wt % of the first biopolymer (e.g., chitosan). In some instances, a scaffold of the disclosure contains no more than about 50 wt %, 55 wt %, 60 wt %, 65 wt %, 70 wt %, 75 wt %, 80 wt %, 85 wt %, 90 wt %, or 95 wt % of the first biopolymer (e.g., chitosan).

In some embodiments, a scaffold of the disclosure contains about, or at least about, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, or 35 wt % chondroitin sulfate. In some embodiments, a scaffold of the disclosure contains less than about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, or 35 wt % chondroitin sulfate.

In some embodiments, a scaffold of the disclosure contains about, or at least about, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, or 35 wt % hyaluronic acid. In some embodiments, a scaffold of the disclosure contains less than about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, or 35 wt % hyaluronic acid.

It may be advantageous in some embodiments for a scaffold to contain a larger weight percent chondroitin sulfate than hyaluronic acid. It can also be advantageous in some embodiments for a scaffold to contain a larger weight percent of the first biopolymer than one or both of chondroitin sulfate and hyaluronic acid.

In one embodiment, the mass ratio of the first biopolymer to hyaluronic acid in the scaffold is about, or at least about 2:1, 3:1, 4:1, 5:1, or 6:1. In one embodiment, the mass ratio of the first biopolymer to hyaluronic acid in the scaffold is less than about 2:1, 3:1, 4:1, 5:1, or 6:1. In some cases, the mass ratio of the first biopolymer to chondroitin sulfate in the scaffold is about, or at least about 3:1, 4:1, 5:1, 6:1, 7:1, 8:1, 9:1, 10:1, 11:1, 12:1, 13:1, or 14:1. In some cases, the mass ratio of the first biopolymer to chondroitin sulfate in the scaffold is less than about 3:1, 4:1, 5:1, 6:1, 7:1, 8:1, 9:1, 10:1, 11:1, 12:1, 13:1, or 14:1. In some instances, the mass ratio of the first biopolymer to the combined mass of hyaluronic acid and chondroitin sulfate in the scaffold is about, or at least about 3:1, 4:1, 5:1, 6:1, 7:1, 8:1, 9:1, 10:1, 11:1, 12:1, 13:1, or 14:1. In some instances, the mass ratio of the first biopolymer to the combined mass of hyaluronic acid and chondroitin sulfate in the scaffold is less than about 3:1, 4:1, 5:1, 6:1, 7:1, 8:1, 9:1, 10:1, 11:1, 12:1, 13:1, or 14:1. In some cases, the mass ratio of chondroitin sulfate to hyaluronic acid in the scaffold is about, or at least about, 1:1, 2:1, 3:1, 4:1, or 5:1. In some cases, the mass ratio of chondroitin sulfate to hyaluronic acid in the scaffold is less than about, 1:1, 2:1, 3:1, 4:1, or 5:1.

In some cases, the scaffold has a thickness of about, or at least about 0.1 mm, 0.2 mm, 0.3 mm, 0.4 mm, 0.5 mm, 0.6 mm, 0.7 mm, 0.8 mm, 0.9 mm, 1.0 mm, 1.25 mm, 1.5 mm, 1.75 mm, 2 mm, 3 mm, 4 mm, 5 mm, 6 mm, 7 mm, 8 mm, 9 mm, 1 cm, 1.5 cm, or 2 cm. In some cases, the scaffold has a thickness of less than about 0.1 mm, 0.2 mm, 0.3 mm, 0.4 mm, 0.5 mm, 0.6 mm, 0.7 mm, 0.8 mm, 0.9 mm, 1.0 mm, 1.25 mm, 1.5 mm, 1.75 mm, 2 mm, 3 mm, 4 mm, 5 mm, 6 mm, 7 mm, 8 mm, 9 mm, 1 cm, 1.5 cm, or 2 cm.

### Cell Delivery

In various aspects, the scaffolds of the disclosure are used to deliver cells to a subject and/or to serve as a reservoir or carrier device for cells. For example, in an embodiment, cells may be delivered to a subject by depositing the cells within the scaffold before or after the scaffold is administered to the subject. The scaffold may serve as a means for holding cells at or near a site of trauma in a patient, or for delaying dispersal of cells to or from a site of trauma. In some cases, the cells assist in wound healing. Non-limiting examples of cells include cells that express and/or secrete bone morphogenetic proteins (BMPs), platelet-rich plasma (PRP) cells, and amniotic cells. In embodiments, the scaffold assists in preventing dispersal of cells away from a site of trauma. Advantageously, the scaffolds can mediate maintaining cells near a site of trauma long enough and at sufficiently high densities to allow the cells to effectively mediate wound healing.

### Antimicrobial Agents

Staphylococcus aureus, Staphylococcus epidermidis, and Pseudomonas aeruginosa are pathogens that are commonly present at musculoskeletal wound sites. S aureus is one cause of osteomyelitis and nongonococcal bacterial arthritis and is often associated with prosthetic joint infection. The disclosure provides scaffolds useful in treating or preventing infection in a wound, complex wound, open fraction, or other site of trauma. Any antimicrobial agent known in the art may be used in the scaffolds of the disclosure at concentrations generally used for such agents.

Antimicrobial agents useful in scaffolds of the disclosure include but are not limited to antibacterials, antifungals, and antivirals. An antimicrobial agent as used herein is an agent that reduces or stabilizes the survival, growth, or proliferation of a pathogen. Antimicrobial agents include but are not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride. Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloridc; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride. In particular embodiments, a scaffold comprises daptomycin.

In one preferred embodiment, a scaffold of the disclosure comprises an agent that treats a multidrug resistant bacteria. In one approach, linezolid may be used to treat multi-drug resistant Gram positive bacteria. Linezolid is commercially available under the trade name Zyvox (Pfizer).

In other embodiments, a scaffold comprises one or more of the following: Benzalkonium Chloride, Cetylpyridinium Chloride, and Chlorhexidine Digluconate. In still other embodiments, a scaffold comprises one or more of antimicrobials: Polyhexamethylene Biguanide, Octenidine Dihydrochloride, Mild Silver Protein, Povidone Iodine (solution or ointment), Silver Nitrate, Silver Sulfadiazine, Triclosan, Cetalkonium Chloride, Myristalkonium Chloride, Tigecycline, Lactoferrin, Quinupristin/dalfopristin, Linezolid, Dalbavancin, Doripenem, Imipenem, Meropenem, and Iclaprim.

In still other embodiments, the scaffold comprises an essential oil having antimicrobial properties. Exemplary essential oils include Oregano oil, tea tree oil, mint oil, sandalwood oil, clove oil, nigella sativa oil, onion oil, leleshwa oil, lavender oil, lemon oil, lemon myrtle oil, neem oil, garlic, eucalyptus oil, peppermint oil, cinnamon oil, and thyme oil.

In still other embodiments, the antimicrobial is a fatty acid (e.g., Cis-2-Decenoic Acid).

Antivirals are agents capable of inhibiting the replication of viruses. Examples of anti-viral agents include but are not limited to 1,-D-ribofuranosyl-1,2,4-triazole-3 carboxamide, 9-2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents useful in scaffolds of the disclosure include fungicidal and fungistatic agents such as, for example, benzoic acid, undecylenic alkanolamide, ciclopirox olamine, polyenes, imidazoles, allylamine, thicarbamates, amphotericin B, butylparaben, clindamycin, econaxole, fluconazole, flucytosine, griseofulvin, nystatin, ketoconazole, and voriconazole. In one preferred embodiment, the antifungal is amphotericin B.

In one embodiment, the disclosure provides scaffolds comprising a combination of one or more antimicrobials and antivirals or antifungals.

### Growth Factors

Growth factors are typically polypeptides or fragments thereof that support the survival, growth, or differentiation of a cell. Such agents may be used to promote wound healing. A scaffold described herein may be used to deliver virtually any growth factor known in the art. Such growth factors include but are not limited to angiopoietin, acidic fibroblast growth factors (aFGF) (GenBank Accession No. NP_149127) and basic FGF (GenBank Accession No. AAA52448), bone morphogenic protein (BMP)(GenBank Accession No. BAD92827), vascular endothelial growth factor (VEGF) (GenBank Accession No. AAA35789 or NP_001020539), epidermal growth factor (EGF)(GenBank Accession No. NP_001954), transforming growth factor α (TGF-α) (GenBank Accession No. NP_003227) and transforming growth factor β (TFG-β) (GenBank Accession No. 1109243A), platelet-derived endothelial cell growth factor (PD-ECGF)(GenBank Accession No. NP_001944), platelet-derived growth factor (PDGF)(GenBank Accession No. 1109245A), tumor necrosis factor α (TNF-α)(GenBank Accession No. CAA26669), hepatocyte growth factor (HGF)(GenBank Accession No. BAA14348), insulin like growth factor (IGF)(GenBank Accession No. P08833), erythropoietin (GenBank Accession No. P01588), colony stimulating factor (CSF), macrophage-CSF (M-CSF)(GenBank Accession No. AAB59527), granulocyte/macrophage CSF (GM-CSF) (GenBank Accession No. NP_000749) and nitric oxide synthase (NOS)(GenBank Accession No. AAA36365). In one preferred embodiment, the growth factor is BMP.

### Analgesics

Scaffolds of the disclosure may be used for the delivery of one or more agents that ameliorate pain, such agents include but are not limited to opioid analgesics (e.g. morphine, hydromorphone, oxymorphone, levorphanol, levallorphan, methadone, meperidine, fentanyl, codeine, dihydrocodeine, oxycodone, hydrocodone, propoxyphene, nalmefene, nalorphine, naloxone, naltrexone, buprenorphine, butorphanol, nalbuphine or pentazocine: a nonsteroidal antiinflammatory drug (NSAID) (e.g., aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, or a pharmaceutically acceptable salt thereof: a barbiturate sedative, e.g. amobarbital, aprobarbital, butabarbital, butabital, mephobarbital, metharbital, methohexital, pentobarbital, phenobarbital, secobarbital, talbutal, theamylal or thiopental or a pharmaceutically acceptable salt thereof: a COX-2 inhibitor (e.g. celecoxib, rofecoxib or valdecoxib.

### Anti-Thrombotics

Scaffolds of the disclosure are also useful for inhibiting, reducing or ameliorating clot formation. In one embodiment, a scaffold contains one or more anti-thrombotics (e.g., thrombin, fibrinogen, cumidin, heparin and calcium salts).

### Anti-Inflammatories

In other embodiments, a scaffold is used to deliver an anti-inflammatory agent. Such anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; omega-3 fatty acids; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

### Delivery of Agents

The disclosure provides a simple means for delivering biologically active agents (e.g., small compounds, nucleic acid molecules, polypeptides) using a scaffold. The scaffold is delivered to a subject and the biologically active agent is eluted from the composition in situ. The scaffold is capable of delivering a therapeutic for the treatment of a disease or disorder that requires controlled and/or localized drug delivery over some period of time (e.g., 1, 3, 5, 7 days: 2, 3, 4 weeks: 1, 2, 3, 6, 12 months). Desirably, the scaffold comprises an effective amount of one or more antibiotics (e.g., amikacin, daptomycin, vancomycin), growth factors that promote wound healing, small molecules, hemostatic agents (e.g., thrombin and/or fibrinogen), anti-thrombotics (e.g., heparin), or cartilage or bone repair agents. The scaffolds are administered in the form of solids, sponges, films, hydrogels, or composites (e.g., sponge fragments in a hydrogel matrix). Such compositions are useful alone or may be used for the delivery of a therapeutic or prophylactic agent delineated herein. Preferably, the scaffold comprises at least about 1 μg, 25 μg, 50 μg, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antibiotic). In another embodiment, the composition releases at least about 1 μg, 25 μg, 50 μg, 100 μg, 250 μg. 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antibiotic) over the course of at least about 1, 2, 3, 4, 5, 6, 7, 8, 9, 10, 14, 21, 28, or 35 days. In still another embodiment, the composition comprises at least about 1 μg, 25 μg, 50 μg, 100 μg, 250 μg, 500 μg, 750 μg, 1 mg, 5 mg. 10 mg, 25 mg, 50 mg, 75 mg, 100 mg, 200 mg, 250 mg. 300 mg, 400 mg, or 500 mg of an agent (e.g., an antibiotic) per cm³.

### Coatings

A biopolymer scaffold of the disclosure (e.g., a scaffold containing chitosan, hyaluronic acid, and/or chondroitin sulfate) may be included in a coating material, such as a film, that is used to coat or wrap a medical device (e.g., drug delivery or other medical device). In embodiments, a scaffold of the disclosure may be a film. In some instances, a scaffold may be used as a wrap (e.g., a nerve wrap, a tendon wrap, an implant wrap, etc.). In some cases, a scaffold may be used to prevent adherence. Such coatings are used, for example, for treating or preventing a pathogen infection or for drug delivery. In orthopedics, many post-surgical infections are associated with implant materials. Patients receiving an orthopedic implant have an infection risk of about 5% for total joint replacements. Bacteria are passively adsorbed on biomaterial surfaces after implantation. The fundamental pathogenic mechanism in biomaterial-centered sepsis is microbial colonization of the biomaterials followed by adjacent damaged tissues. Patients that suffer from such infections often require the removal and replacement of the implant to eradicate the infection.

To treat or prevent an implant-associated infection a scaffold of the disclosure is applied to the medical device (e.g., implant). The scaffold provides for release of a therapeutic or prophylactic agent from the device. Such agents advantageously reduce the risk of infection associated with conventional implants. Such coatings may be applied to any medical device known in the art, including, but not limited to orthopedic devices (e.g., for joint implants, fracture repairs, spinal implants, screws, rods, plates); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); wound management devices; drug-delivering vascular stents (e.g., a balloon-expanded stents); other vascular devices (e.g., grafts, catheters, valves, artificial hearts, heart assist devices); implantable defibrillators; blood oxygenator devices (e.g., tubing, membranes); membranes; biosensors; shunts for hydrocephalus; endoscopic devices; infection control devices; dental devices (e.g., dental implants, fracture repair devices), urological devices (e.g., penile, sphincter, urethral, bladder and renal devices, and catheters); colostomy bag attachment devices; ophthalmic devices (e.g. intraocular coils/screws); glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts), urinary catheters, intravenous catheters, small diameter grafts, vascular grafts, artificial lung catheters, atrial septal defect closures, electro-stimulation leads for cardiac rhythm management (e.g., pacer leads), glucose sensors (long-term and short-term), degradable coronary stents (e.g., degradable, non-degradable, peripheral), blood pressure and stent graft catheters, birth control devices, prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, urological catheters/stents/implants, in vitro diagnostics, aneurysm exclusion devices, and neuropatches.

Examples of other suitable devices include, but are not limited to, vena cava filters, urinary dialators, endoscopic surgical tissue extractors, atherectomy catheters, clot extraction catheters, coronary guidewires, drug infusion catheters, esophageal stents, circulatory support systems, angiographic catheters, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

It is noted that in other embodiments of the present disclosure, the scaffold of the present disclosure may self-adhere to the medical device or may be adhered to the device by means other than coating materials, such as adhesives, sutures, or compression. Any suitable method know in the art may be utilized to adhere the scaffold to a surface. For example, the scaffold may be adhered to the surface by pressing the scaffold onto the device, wrapping the device with a chitosan film, or spraying a scaffold onto the device.

The scaffolds with biocompatible surfaces may be utilized for various medical applications including, but not limited to, drug delivery devices for the controlled release of pharmacologically active agents, including wound healing devices, such as dressings, suture material and meshes, medical device coatings/films and other biocompatible implants. The methods of the disclosure would also provide for the controlled degradation of the coating to a suitable timeframe for release of active components.

### Wound Healing Devices

The present disclosure provides wound healing devices containing a biopolymer scaffold of the disclosure (e.g., scaffolds comprising chitosan, hyaluronic acid, and/or chondroitin sulfate). In embodiments, the devices and scaffolds of the disclosure are suitable for use in dermal regeneration. Not intending to be bound by theory, the porous nature of the device allows for cellular infiltration to aid in regrowth of tissue and neovascularization.

In some embodiments, the wound healing device contains a scaffold to which a polymer film has been affixed. In some cases, the polymer film is affixed to a surface (e.g., a planar surface) of the scaffold. In some cases, it is advantageous to affix the polymer film to a portion of the scaffold otherwise exposed to an external environment when inserted into a wound. The polymer film may be perforated or meshed to allow for gasses, such as oxygen and/or water vapor, and/or liquids to penetrate the film. The polymer film may be completely occlusive. The film may have any suitable thickness, such as at least about, or about 0.01 mm, 0.02 mm, 0.05 mm, 0.06 mm, 0.07 mm, 0.08 mm, 0.09 mm, 0.10 mm, 0.20 mm, 0.30 mm, 0.40 mm, 0.50 mm. 0.60 mm, 0.70 mm, 0.80 mm, 0.90 mm, 1 mm, 2 mm, 3 mm, 4 mm, or 5 mm. The film may have a thickness of less than about 0.01 mm, 0.02 mm, 0.05 mm, 0.06 mm, 0.07 mm, 0.08 mm, 0.09 mm, 0.10 mm, 0.20 mm, 0.30 mm, 0.40 mm, 0.50 mm. 0.60 mm, 0.70 mm, 0.80 mm, 0.90 mm, 1 mm, 2 mm, 3 mm, 4 mm, or 5 mm. In some instances, the film is manufactured of polyurethane, silicone, or any other suitable polymer. The polymer film is typically attached to the scaffold using an adhesive, such as an acrylic adhesive.

In embodiments, the polymer film improves a characteristic of the wound healing device, such as the structural integrity of the device, ability to protect a wound from environmental contamination, and/or control the moisture content of the scaffold. In other embodiments, the wound healing device and/or polymer film is fenestrated. In some embodiments, the polymer backing does not extend beyond a perimeter of a face of the scaffold. In other embodiments, the backing does extend beyond the perimeter of the scaffold.

In some embodiments, the scaffold is applied directly to a wound and stapled in place.

The wound healing devices may be configured by forming the scaffold into a shape and size sufficient to accommodate the wound being treated. If desired, the wound healing device comprises chitosan fibers. Wound healing devices are desirably produced in whatever shape and size is necessary to provide optimum treatment to the wound. These devices may be produced in forms that include, but are not limited to, plugs, meshes, strips, sutures, dressings, or any other form able to accommodate and assist in the repair of a wound. The damaged portions of the patient that may be treated with devices made of the scaffold of the present disclosure include, but are not limited to, bone, cartilage, skin, muscle and other tissues (nerve, brain, spinal cord, heart, lung). Other similar devices are administered to assist in the treatment repair and remodeling of a damaged tissue, bone, or cartilage. For some applications, it is desirable for the device to be incorporated into an existing tissue to facilitate wound repair. For other applications, it is desirable for the device to degrade over the course of days, weeks, or months. Such degradation may be advantageously tailored to suit the needs of a particular subject using the methods described herein. The elution and/or degradation profile of a scaffold may be altered as described herein by modulating the following variables: degree of deacetylation, neutralization solution, solvent make-up, and chitosan weight %, molecular weight, and/or crystallinity.

If desired the scaffold is loaded with agents and the scaffold is delivered to a wound to form a delivery system for the agent. Preferably, the scaffold contains an effective amount of a chemical or pharmaceutically active component. In one embodiment, the scaffold self-adheres to a site at which delivery is desired. In another embodiment, an adhesive or other adhering means may be applied to the outer edges of the scaffold to hold the scaffold in position during the delivery of the chemical or pharmaceutically active component. Such adherent means may be used alone or in combination with the self-adhering properties of components of the scaffold (e.g., chitosan). Scaffolds of the disclosure may provide for the local administration of a desired amount of a therapeutic agent.

In other embodiments, the scaffold is administered directly to an injured area. In some instances, the injured area includes at least a portion of the dura. A scaffold of the may be administered by sprinkling, packing, implanting, mechanical securing (e.g., by stapling or suturing), inserting or applying or by any other administration means to a site of trauma (e.g., open wound, open fracture, complex wound).

In some embodiments, a scaffold is administered to, is administered to replace an injured portion of, or is administered to facilitate repair of the dura. In some cases, a scaffold of the disclosure is used as a dura repair device.

### Delivery

Biopolymer scaffolds of the disclosure (e.g., scaffolds containing chitosan, hyaluronic acid, and/or chondroitin sulfate) may be delivered by any method known to the skilled artisan. In one approach, a scaffold is locally delivered to a site of trauma in the form of a film or sponge. The film, sponge, or other wound management device may be configured to fit a wound of virtually any size. In another approach, the scaffold is surgically implanted at a site where promotion of healing, treatment, and/or prevention of infection is required. If desired, the scaffold is loaded with one or more antibiotics or other biologically active agents (e.g., an iodine compound) by a clinician within the surgical suite where treatment is to be provided. This advantageously allows the scaffold to be loaded with a specific agent or combination of agents tailored to the needs of a particular patient at the point at which care is to be provided.

### Screening Assays

As described herein, the present disclosure provides for the delivery of therapeutic or prophylactic agents to wounds in vivo. The disclosure is based in part on the discovery that therapeutic agents may be delivered using a scaffold where the agents and degradation of the scaffold is tailored to suit the needs of a particular patient. To identify scaffolds having the desired degradation and elution profiles, screening may be carried out using no more than routine methods known in the art and described herein. For example, scaffolds are loaded with one or more therapeutic agents and such compositions are subsequently compared to untreated control compositions to identify scaffolds that promote healing. In another embodiment, the degradation of a scaffold of the disclosure is assayed in vivo to identify the degree of deacetylation that corresponds to the desired degradation profile. Any number of methods are available for carrying out screening assays to identify such scaffolds.

In one working example, candidate compounds are added at varying concentrations to a scaffold. The degree of infection or wound healing is then measured using standard methods. The degree of infection (e.g., number of bacteria) or wound healing in the presence of the compound is compared to the level measured in a control lacking the compound. A compound that enhances healing is considered useful in the disclosure: such a compound may be used, for example, as a therapeutic to prevent, delay, ameliorate, stabilize, or treat a disease described herein (e.g., tissue damage). In other embodiments, the compound prevents, delays, ameliorates, stabilizes, or treats a disease or disorder described herein. Such therapeutic compounds may be useful in vivo.

In another approach, scaffolds containing a chitosan having varying degrees of deacetylation are incubated in vivo, added to a wound, or are contacted with a composition comprising an enzyme having chitosan-degrading activity. The length of time required for chitosan degradation is then measured using standard methods as described herein. A scaffold having the desired degradation profile (e.g., degrading in 3 days, 5 days, 1 week, 2 weeks, 3 weeks, 1 month, 2 months, 3 months) is considered useful in the disclosure: such a scaffold may be used, for example, as a therapeutic to prevent, delay, ameliorate, stabilize, or treat a disease described herein (e.g., tissue damage). In other embodiments, the scaffold prevents, delays, ameliorates, stabilizes, or treats a disease or disorder described herein. Such therapeutic scaffolds are useful in vivo.

The present disclosure provides methods of treating pathogen infections (e.g., bacterial, viral, fungal), complex wounds, open fractures, trauma, and associated diseases and/or disorders or symptoms thereof which comprise administering a therapeutically effective amount of a scaffold and a therapeutic or prophylactic agent to a subject (e.g., a mammal, such as a human). Thus, one embodiment is a method of treating a subject suffering from or susceptible to an infection, trauma, wound, open fracture, or related disease or disorder that requires targeting of a therapeutic composition to a site. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a scaffold described herein to produce such effect. Identifying a subject in need of such treatment can be in the judgment of a subject or a health care professional and can be subjective (e.g., opinion) or objective (e.g., measurable by a test or diagnostic method).

The therapeutic methods of the disclosure (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for an infection, in need of healing, having a trauma, wound, open fracture, or related disease, disorder, or symptom thereof. Determination of those subjects “at risk” can be made by any objective or subjective determination by a diagnostic test or opinion of a subject or health care provider (e.g., genetic test, enzyme or protein marker, family history, and the like). The agents herein may be also used in the treatment of any other disorders in which it is desirable to promote healing or treat or prevent an infection.

In one embodiment, the disclosure provides a method of monitoring treatment progress. The method includes the step of determining a level of diagnostic marker (Marker) (e.g., wound healing parameters, number of bacterial cells, or any target delineated herein modulated by a compound herein, C-reactive protein, cytokine levels, or indicator thereof, etc.) or diagnostic measurement (e.g., screen, assay) in a subject suffering from or susceptible to an infection, disorder or symptoms thereof, in which the subject has been administered a therapeutic amount of a composition (e.g., using a scaffold containing a therapeutic or prophylactic agent) herein sufficient to treat the disease or symptoms thereof. The level of Marker determined in the method may be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this disclosure: this pre-treatment level of Marker can then be compared to the level of Marker in the subject after the treatment commences, to determine the efficacy of the treatment.

### Test Compounds and Extracts

In general, therapeutic compounds suitable for delivery from a scaffold of the disclosure are known in the art or are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the disclosure. Compounds used in screens may include known compounds (for example, known therapeutics used for other diseases or disorders). Alternatively, virtually any number of unknown chemical extracts or compounds may be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds may be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989): T. W. Greene and P. G. M. Wuts, Protective Groups in Organic Synthesis, 2nd ed., John Wiley and Sons (1991): L. Fieser and M. Fieser, Fieser and Fieser's Reagents for Organic Synthesis, John Wiley and Sons (1994): and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries may be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is identified as containing a compound of interest, further fractionation of the positive lead extract is necessary to isolate chemical constituents responsible for the observed effect. Thus, the goal of the extraction, fractionation, and purification process is the careful characterization and identification of a chemical entity within the crude extract that achieves a desired biological effect. Methods of fractionation and purification of such heterogenous extracts are known in the art.

### Kits

The disclosure provides kits that include scaffolds and wound healing devices of the disclosure (e.g., scaffolds comprising chitosan, hyaluronic acid, and/or chondroitin sulfate). In one embodiment, the kit includes two packets, one comprising a biopolymer scaffold and the second a packet containing a therapeutic or prophylactic agent that that prevents or treats infection (e.g., an antimicrobial agent, such as iodine) or that promotes healing (e.g., growth factor, anti-inflammatory, clot promoting agent, anti-thrombotic, etc., and/or cells, such as stem cells, platelet-rich plasma (PRP) cells, etc.). In other embodiments, the kit contains a therapeutic device, such as a scaffold or wound healing device useful in wound healing. If desired, the aforementioned scaffold or wound healing device further comprise an agent described herein (e.g., iodine). In an embodiment, the scaffold is part of a kit suitable for use in the collection of autologous cells from a subject. In an embodiment, the scaffold is part of a kit for delivering cells to a subject, where the scaffold is loaded with cells prior to or subsequent to administration to a subject.

In some embodiments, a kit of the disclosure contains a scaffold prepared according to the methods provided herein and a packet containing an iodine solution. In embodiments, the methods of the disclosure involve rupturing, cutting, or piercing the packet containing the iodine solution at a point of treatment and contacting a scaffold with the solution so that the scaffold becomes partially or fully saturated with the iodine solution. In embodiments, the kit contains the packet containing the iodine solution and the scaffold both separately contained within a larger packet so that rupturing (e.g., through providing a force on the packet until the iodine solution is released from the packet), cutting, or puncturing of the iodine packet leads to the iodine solution contacting and being absorbed in part or fully by the scaffold within the larger packet prior to removing the scaffold from the larger packet and administering the scaffold to a subject. In various embodiments, the iodine solution may be replaced with any solution containing any agent described herein, or combinations thereof. In some instances, the kit contains a scaffold and a container containing an agent described herein so that the agent may be applied to the scaffold at a site of care.

In some embodiments, the kit comprises a sterile container which contains a scaffold or wound healing device of the disclosure: such containers may be boxes, ampoules, bottles, vials, tubes, bags, pouches, blister-packs, or other suitable container forms known in the art. Such containers may be made of plastic, glass, laminated paper, metal foil, or other materials suitable for holding medicaments.

If desired a scaffold of the disclosure is provided together with instructions for using it in a prophylactic or therapeutic method described herein. The instructions will generally include information about the use of the composition for the treatment of a trauma, infection, or related disease in a subject in need thereof. The instructions may be printed directly on the container (when present), or as a label applied to the container, or as a separate sheet, pamphlet, card, or folder supplied in or with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989): “Oligonucleotide Synthesis” (Gait, 1984): “Animal Cell Culture” (Freshney, 1987): “Methods in Enzymology” “Handbook of Experimental Immunology” (Weir, 1996): “Gene Transfer Vectors for Mammalian Cells” (Miller and Calos, 1987); “Current Protocols in Molecular Biology” (Ausubel, 1987): “PCR: The Polymerase Chain Reaction”, (Mullis, 1994): “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Dissolving Chitosan, Chondroitin Sulfate, and Hyaluronic Acid Together in Solution

Experiments were undertaken to develop a method for dissolving chitosan, chondroitin sulfate, and hyaluronic acid in an aqueous solution. Such solutions may be used for the preparation of porous biopolymer scaffolds containing two or more of the three compounds.

First experiments were undertaken to prepare solutions of chondroitin sulfate and/or hyaluronic acid dissolved in water. Initially when trying to dissolve either the chondroitin sulfate and/or hyaluronic acid into slightly acidic solution, the compounds coagulated densely and would not dissolve. Next, a basic solution was tried and, again, the compounds coagulated and would not dissolve. Finally, a pH neutral solution (pH between 6 and 8) was used and the polymers, both individually and in combination, dissolved in solution and produced a viscous gel.

Next, experiments were undertaken to dissolve chitosan in the solutions containing dissolved chondroitin sulfate and hyaluronic acid. Chitosan was added to the solutions, and the chitosan floated on the surface or within the solution. Following the addition of the chitosan, lactic acid and acetic acid were added to the solution to lower the pH (e.g., to a value between 4 and 6). The addition of the acids enabled the dissolution of chitosan in the solutions containing chondroitin sulfate and hyaluronic acid, which yielded a viscous gel. The final concentration of total acid in the in the solution containing dissolved chondroitin sulfate, hyaluronic acid, and chitosan, was between about 0.5% and 1.0% v/v, where the solution contained equal volumetric percents of lactate and acetate.

### Example 2: Preparation of a Porous Biopolymer Scaffold Containing Chondroitin Sulfate and Chitosan

Having established a method for dissolving chitosan together with chondroitin sulfate and/or hyaluronic acid in a solution, an experiment was undertaken to prepare a porous biopolymer scaffold containing chondroitin sulfate and chitosan.

First, 7200 ml of deionized water with a pH of 7 at ambient temperature was heated to about 37° C. in a 10.7 L pot and mixed using a 4-inch dispersion blade. Then, 14.4 g chondroitin sulfate (0.2% m/v final concentration) was added with continuous mixing. Next, 46.8 g chitosan (Chitopharm L™) (0.65% m/v final concentration) was added with continuous mixing. Then, 27 mL each of an aqueous solution of DL-lactic acid (0.375% v/v concentration) and an aqueous solution of acetic acid (0.375 v/v concentration) were added with continuous mixing until the chitosan was fully dissolved. The final combined concentrations of the two acids was 0.75% v/v (50/50 split with lactic acid and acetic acid). The solution was mixed at high speeds for 30 minutes. Following mixing, the solution was pumped into 10 700 mL trays using a peristaltic pump and ⅜ inch tubing. The solutions were then lyophilized.

For neutralization, each biopolymer scaffold was soaked in about 1000 mL 0.5 N NaOH for 15-20 second. The scaffolds were then added to 60 qt stainless steel vats filled with deionized water where the water was mixed continuously. The biopolymer scaffolds were then lifted out of the water with strainers and the pH of the water was adjusted to about 5.0 using acetic acid. The scaffolds were then raised and lowered into the water to circulate the water through the biopolymer scaffolds. The process of adjusting the pH and raising and lowering the biopolymer scaffolds was continued until the final pH of the scaffolds was between 6.0 and 8.0. The scaffolds were then lyophilized again and die-cut to yield porous biopolymer scaffolds about 1.25 mm thick and either about 5 cm×5 cm, 10 cm×12.5 cm, or 20 cm×25 cm. The scaffolds were subsequently each individually packaged in sterile pouches.

### Example 3: Batch 2 Preparation of a Porous Biopolymer Scaffold Containing Chondroitin Sulfate, Hyaluronic Acid, and Chitosan

An experiment was undertaken to prepare a second batch of porous biopolymer scaffold containing chondroitin sulfate, hyaluronic acid and chitosan.

First, 43 L of deionized water with a pH of 7 at ambient temperature was heated to about 37° C. in a 60 quart pot and mixed using a 4-inch dispersion blade. Then, 21.5 g sodium hyaluronate (HA) (0.5 g/L final concentration) was added with continuous mixing. Then, 43 g chondroitin sulfate (1 g/L final concentration) was added with continuous mixing. Next, 301 g chitosan (Chitopharm L™) (7 g/L final concentration) was added with continuous mixing. Then, 215 mL each of an aqueous solution of DL-lactic acid (0.5% v/v concentration) and an aqueous solution of acetic acid (0.5% v/v concentration) were added together with continuous mixing until the chitosan was fully dissolved. The final combined concentrations of the two acids was 1% v/v (50/50 split with lactic acid and acetic acid). The solution was mixed at high speeds until all components were evenly distributed in the solution and fully dissolved. Following mixing, the solution was pumped into 60 700 mL trays using a peristaltic pump and ⅜ inch tubing. The solutions were then lyophilized.

For neutralization, each biopolymer scaffold was soaked in about 1000 mL 0.5 N NaOH for 15-20 second. The scaffolds were then added to 60 qt stainless steel vats filled with deionized water where the water was mixed continuously. The biopolymer scaffolds were then lifted out of the water with strainers and the pH of the water was adjusted to about 5.0 using acetic acid. The scaffolds were then raised and lowered into the water to circulate the water through the biopolymer scaffolds. The process of adjusting the pH and raising and lowering the biopolymer scaffolds was continued until the final pH of the scaffolds was between 6.0 and 8.0. The scaffolds were then lyophilized again and die-cut to yield porous biopolymer scaffolds about 1.25 mm thick and either about 5 cm×5 cm, 10 cm×12.5 cm, or 20 cm×25 cm. The scaffolds were subsequently each individually packaged in sterile pouches.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

This application includes subject matter that may be related to subject matter described in International Application Publications No. WO2010107794, WO2014142915, WO2015123501, WO2010123989; in U.S. Pat. No. 5,489,304; or in U.S. Patent Application Publication No. 2011/0129515, the disclosures of each of which are herein incorporated by reference in their entireties for all purposes.

All patents and publications mentioned in this specification are herein incorporated by reference to the same extent as if each independent patent and publication was specifically and individually indicated to be incorporated by reference.

What is claimed is:

1. A biopolymer scaffold comprising chondroitin sulfate and/or hyaluronic acid and chitosan, collagen, and/or alginate, wherein if the biopolymer scaffold contains chondroitin sulfate and not hyaluronic acid, then the biopolymer scaffold does not contain collagen.

2. The biopolymer scaffold of claim 1, comprising hyaluronic acid and chitosan.

3. The biopolymer scaffold of claim 1, comprising chondroitin sulfate, and chitosan.

4. The biopolymer scaffold of claim 1, comprising chitosan, hyaluronic acid, and chondroitin sulfate.

5. The biopolymer scaffold of claim 1, wherein the scaffold comprises at least about 50% or more by weight chitosan, collagen, and/or alginate.

6. The biopolymer scaffold of claim 1, wherein the scaffold comprises at least about 75% or more by weight chitosan, collagen, and/or alginate.

7. The biopolymer scaffold of claim 1, wherein the scaffold comprises about 5-30% by weight chondroitin sulfate and/or about 1-10% hyaluronic acid.

8. The biopolymer scaffold of claim 1, wherein the scaffold comprises from about 10-25% chondroitin sulfate and/or about 3-8% hyaluronic acid.

9. A multilayered medical device comprising the biopolymer scaffold of claim 1 and a polymer film.

10. A method for producing a biopolymer scaffold, the method comprising:
(a) dissolving chondroitin sulfate and/or hyaluronic acid in an aqueous solution at about neutral pH;
(b) adding to the solution chitosan, collagen, and/or alginate and a combined volumetric percent of about 0.25% to about 2.0% of lactate and acetate;
(c) lyophilizing the aqueous solution of (b) to produce a biopolymer scaffold;
(d) adjusting the pH of the biopolymer scaffold to a pH between about 6.0 to about 8.0; and
(e) lyophilizing the biopolymer scaffold of (d) to produce a biopolymer scaffold.

11. The method of claim 10, wherein step (b) comprises adding approximately equal volumetric percentages lactic acid and/or acetic acid.

12. The method of claim 10, wherein the aqueous solution of (a) comprises 0.01-5% weight/weight (w/w) chondroitin sulfate and/or hyaluronic acid.

13. The method of claim 10, wherein the aqueous solution of (a) comprises 0.2% to 5% w/w chondroitin sulfate and/or hyaluronic acid.

14. The method of claim 10, wherein the aqueous solution of (a) comprises 0.1-5.0% chitosan, collagen, and/or alginate.

15. The method of claim 10, wherein the aqueous solution of (a) comprises about 0.5-1% chitosan, collagen, and/or alginate.

16. The method of claim 10, wherein the method further comprises (f) contacting the lyophilized biopolymer scaffold with an aqueous solution comprising a therapeutic agent followed by (g) lyophilizing the scaffold again.

17. The method of claim 16, wherein the therapeutic agent is selected from the group consisting of an anti-microbial agent, a growth factor, an hemostatic agent, an antithrombotic agent, cells, and an analgesic agent.

18. The method of claim 10, wherein the lyophilization step of (c) or (e) reduces the amount of water within the scaffold to less than about 5-10% of the weight of the scaffold.

19. The method of claim 18, wherein the lyophilization step of (c) or (e) reduces the amount of water within the scaffold to less than about 1% of the weight of the scaffold.

20. A biopolymer scaffold produced by the method of claim 10.

21. A method for treating a site of trauma in a subject, the method comprising contacting the site with the biopolymer scaffold of claim 1.

22. The method of claim 21, wherein the site of trauma is a burn or wound.

23. The method of claim 21, wherein the biopolymer scaffold is applied topically to the site of trauma.

24. The method of claim 21, wherein the biopolymer scaffold increases cellular infiltration of the site of trauma relative to a corresponding control subject that received conventional therapy.

25. The method of claim 21, wherein the biopolymer scaffold is infiltrated by cells at the site of trauma regrowth of tissue and neovascularization.

26. A kit for use in treatment of a wound comprising the biopolymer scaffold of claim 1.

27. The kit of claim 26, further comprising a therapeutic agent.

28. The kit of claim 27, wherein the therapeutic agent is selected from the group consisting of an anti-microbial, a growth factor, a hemostatic agent, an antithrombotic agent, cells, and an analgesic agent.

29. The kit of claim 28, wherein the anti-microbial is an iodine solution.

30. The kit of claim 28, wherein the biopolymer scaffold and the anti-microbial are formulated separately and combined at a point of treatment.
